(19) United States
(12) Patent Application Publication
(10) Pub. No.: US 2003/0053967 A1
(43) Pub. Date: Mar. 20, 2003

(54) Use of 3,4-dihydroxy-mandelic acid for protecting against oxidative damage caused by ultra-violet light

(76) Inventors: Jakob Peter Ley, Holzminden (DE); Roland Langner, Bevern (DE); William Johncock, Hoxter (DE)

Correspondence Address: BAYER CORPORATION, PATENT DEPARTMENT, 100 BAYER ROAD, PITTSBURGH, PA, 15205, US

(21) Appl. No.: 10/221,707

(22) PCT Filed: Mar. 7, 2001
(86) PCT No.: PCT/EP01/02546

(30) Foreign Application Priority Data
Mar. 18, 2000 (DE) 100 13 578.1

(51) Int. Cl. A61K 31/192; A61K 7/42

(52) U.S. Cl. 424/59; 514/570

(57) ABSTRACT

The invention relates to the use of 3,4-dihydroxy-mandelic acid as an anti-oxidant and/or free-radical scavenger for protecting the skin, in particular human skin against oxidative damage caused by ultra-violet light and also for protecting cosmetic or pharmaceutical preparations, other objects and foodstuffs against oxidation and/or photo-oxidation.

The invention relates to the use of 2-(3,4-dihydroxyphenyl)-2-hydroxyacetic acid, called 3,4-dihydroxymandelic acid below, as antioxidant and/or free-radical scavenger for protecting against ultraviolet light-induced oxidative damage.

Ultraviolet light, in particular ultraviolet light in the range from 290 to 400 nm, triggers photooxidative processes on or in the skin of, for example, mammals or humans, various reactive oxygen compounds or free radicals being formed starting from oxygen. These may, for example, damage or destroy intracellular molecules and thus weaken the vitality of the cell or even cause it to die. In addition, the reactive oxygen compounds or free radicals may also damage the intracellular molecules or structures. In the case of the skin, the lipid layer, which serves as a barrier against the environment, and the sebum, in particular, can be destroyed by oxidative processes. A main constituent of the vital sebum is the greatly unsaturated and very oxidation-sensitive squalene.

Antioxidants or free-radical scavengers, which aid the natural defense mechanisms against free radicals and reactive oxygen compounds in physiological systems, protect the lipid layer of the skin against oxidative processes. They also protect the oxidation-sensitive constituents of cosmetics, pharmaceuticals or foods against autoxidation.

Antioxidants are substances which, in small concentrations relative to the oxidizable substrate significantly delay oxidation, or prevent it entirely. Many antioxidants also function as free-radical scavengers and/or as complexing agents for heavy metal ions.

The object of the present invention is to develop active ingredients which, because of their strong specific free-radical scavenging and/or antioxidative action, are able to protect cells and tissue of the skin of mammals against the harmful influences of free radicals and oxidative processes.

The invention thus relates to the use of 3,4-dihydroxymandelic acid including its stereoisomers or mixtures thereof as antioxidants and/or free-radical scavengers for protecting the skin of mammals, but in particular human skin, against oxidative processes which are induced by ultraviolet light.

For use, the 3,4-dihydroxymandelic acid is applied in the form of topical preparations, preferably in the form of cosmetic or dermatological preparations, to the skin and/or the hair in an effective amount.

Surprisingly, it has now been found that the use of 3,4-dihydroxymandelic acid according to the invention including its stereoisomers or mixtures thereof suppresses particularly well the harmful influences of free radicals and oxidative processes which are induced by UV light on or in the skin and aids the natural antioxidative processes. In particular, the use of 3,4-dihydroxymandelic acid including its stereoisomers or mixtures thereof in combination with other antioxidants suppresses particularly well the harmful influences of free radicals and oxidative processes which are induced by UV light on or in the skin and aids the natural antioxidative processes.

In addition to many other α-hydroxycarboxylic acids, 3,4-dihydroxymandelic acid has been proposed in U.S. Pat. No. 5,834,510 as a skin care, nail care and hair care agent. In this patent specification, α-hydroxycarboxylic acids, among which 3,4-dihydroxy-mandelic acid is not mentioned, are used in concentrations greater than or equal to 2% by weight, preferably greater than or equal to 7% by weight, based on the total volume of the formulation. U.S. Pat. No. 5,834,510 does not describe that 3,4-dihydroxy-mandelic acid including its stereoisomers and mixtures thereof have an antioxidative or free-radical scavenging action.

It was therefore surprising that 3,4-dihydroxymandelic acid including its stereoisomers and mixtures thereof in concentrations of less than 1% by weight, preferably 0.001 to 0.5% by weight, based on the total weight of a formulation, has an exceptionally strong antioxidative and free-radical scavenging action. Its excellently protecting action of the ultraviolet light-initiated oxidation of the sebum of human skin, even at a concentration of 0.1% by weight, based on the total weight of a formulation, could not have been foreseen.

In addition, it has been found that the combination of 3,4-dihydroxymandelic acid including its stereoisomers and mixtures thereof with at least one further antioxidant, preferably at least one lipophilic antioxidant, displays an unexpectedly strong synergistic action.

According to the invention, further antioxidants which may be used are all antioxidants customary or suitable for cosmetic and/or dermatological applications. The antioxidants are advantageously chosen from the group of imidazoles (e.g. urocanic acid) and derivatives thereof, carotenoids, carotenes (e.g. α-carotene, β-carotene, lycopene) and derivatives thereof, chlorogenic acid and derivatives thereof, lipoic acid and derivatives thereof, aurothioglucose, propylthiouracile and other thiols (e.g. thioredoxin, glutathione, cysteine, cystine, cystamine and the glycosyl and N-acyl derivatives thereof and alkyl esters thereof), and salts thereof, dilauryl thiodipropionate, distearyl thiodipropionate, thiodipropionic acid and derivatives thereof, and phenolic acid amides of phenolic benzylamine (e.g. homovanillic acid, 3,4-dihydroxyphenylacetic acid, ferulic acid, sinapic acid, caffeic acid, dihydroferulic acid or dihydrocaffeic acid of 3,4-dihydroxybenzylamine, 2,3,4-trihydroxybenzylamine and 3,4,5-trihydroxybenzylamine), catechol oximes or catechol oxime ethers (e.g. 3,4-dihydroxybenzaldoxime, 3,4-dihydroxyaceto-phenone oxime or 3,4-dihydroxybenzaldehyde O-ethyloxime), and also (metal) chelating agents (e.g. 2-hydroxy fatty acids, phytic acid, lactoferrin), humic acid, bile acids, bile extracts, bilirubin, biliverdin, folic acid and derivatives thereof, ubiquinone and ubiquinol and derivatives thereof, vitamin C and derivatives thereof (e.g. ascorbyl palmitate, Mg ascorbyl phosphate, ascorbyl acetate), tocopherols and derivatives (e.g. vitamin E acetate), vitamin A and derivatives (e.g. vitamin A palmitate), rutinic acid and derivatives thereof, flavonoids (e.g. quercetin, α-glucosylrutin) and derivatives thereof, phenolic acids (e.g. gallic acid, ferulic acid) and derivatives thereof (e.g. propyl gallate, ethyl gallate, octyl gallate), furfurylideneglucitol, butylhydroxytoluene, butylhydroxyanisole, tert-butylhydroquinone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, resveratrol) and derivatives of these said active ingredients which are suitable according to the invention.

3,4-Dihydroxymandelic acid including its stereoisomers or mixtures thereof can be incorporated into customary cosmetic or dermatological preparations. The amount of 3,4-dihydroxymandelic acid including its stereoisomers or mixtures thereof in the cosmetic or dermatological preparations is 0.001% by weight to 1% by weight, preferably 0.001% by weight to 0.5% by weight, based on the total weight of the preparations. The cosmetic or dermatological preparations may here be in the form of “water-in-oil” emulsions, “oil-in-water” emulsions, “water-in-oil-in-water” emulsions or “oil-in-water-in-oil” emulsions, microemulsions, gels, soaps, solutions, e.g. in oils, alcohols or silicone oils, sticks, aerosols, sprays and also foams. Further customary cosmetic auxiliaries and additives may be present in amounts of from 0.001-99.999% by weight, preferably 1-99% by weight, based on the total weight of the formulation. In addition, the formulations may have water in an amount up to 99.999% by weight, preferably 1 to 99% by weight, based on the total weight of the formulation.

The amount of further antioxidants, where one or more compounds may be used, in the cosmetic or dermatological preparations is preferably 0.001 to 10% by weight, preferably 0.001 to 5% by weight, particularly preferably 0.001 to 1% by weight, based on the total weight of the preparations.

To prepare the cosmetic and dermatological preparations, in a further embodiment, 3,4-dihydroxymandelic acid including its stereoisomers and mixtures thereof can also be incorporated beforehand into liposomes, e.g. starting from phosphatidylcholine, into microspheres, into nanospheres or else into capsules made of a suitable matrix, for example made of natural waxes such as, for example, beeswax, candellila wax, carnauba wax or synthetic waxes such as, for example, silicone waxes, paraffin waxes or made of proteins such as, for example, gelatin or from polysaccharides such as, for example, agar, hyaluronic acid, starch, cellulose or chitin.

The cosmetic and dermatological preparations comprise cosmetic auxiliaries and additives as are customarily used in such preparations, e.g. sunscreens (e.g. organic or inorganic UV sunscreen filters, preferably micropigments), preservatives, bactericides, fungicides, virucides, cooling active ingredients, plant extracts, antiinflammatory active ingredients, substances which accelerate wound healing (e.g. chitin or chitosan and derivatives thereof), film-forming substances (e.g. polyvinylpyrrolidones or chitosan or derivatives thereof), vitamins (e.g. vitamin C and derivatives, tocopherols and derivatives, vitamin A and derivatives), 2-hydroxycarboxylic acids (e.g. citric acid, maleic acid, L-, D- or dl-lactic acid), perfumes, antifoams, dyes, pigments which have a coloring action, thickeners, surface-active substances, emulsifiers, emollients, moisturizers and/or humectants (e.g. glycerol or urea), fats, oils, unsaturated fatty acids or derivatives thereof (e.g. linoleic acid, α-linolenic acid, γ-linolenic acid or arachidonic acid and their respective natural or synthetic esters), waxes or other customary constituents of a cosmetic or dermatological formulation, such as alcohols, polyols, polymers, foam stabilizers, electrolytes, organic solvents, silicone derivatives or chelating agents (e.g. ethylenediaminetetraacetic acid and derivatives thereof).

The amounts of cosmetic or dermatological auxiliaries and additives and perfume to be used in each case can be readily determined by a person skilled in the art by simple experimentation, depending on the nature of the product in question.

For the use according to the invention, the cosmetic and dermatological preparations are applied to the skin and/or the hair in a sufficient amount in the manner customary for cosmetics.

The cosmetic and dermatological preparations can, however, also comprise UVA and/or UVB filter substances, where the total amount of filter substances may be 0.1 to 30% by weight, preferably 0.5 to 10% by weight, based on the total weight of the preparations, giving, for example, sunscreens for skin and hair. UV filter substances which may be used are, for example, 3-benzylidenecamphor derivatives (e.g. 3-(4-methylbenzylidene)-dl-camphor), aminobenzoic acid derivatives (e.g. 2-ethylhexyl 4-(N,N-dimethylamino)benzoate or menthyl anthranilate), 4-methoxycinnamates (e.g. 2-ethylhexyl p-methoxycinnamate or isoamyl p-methoxycinnamate), benzophenones (e.g. 2-hydroxy-4-methoxybenzophenone), mono- or polysulfonated UV filters [e.g. 2-phenylbenzimidazole-5-sulfonic acid, sulisobenzones or 1,4-bis(benzimidazolyl)-benzene-4,4′, 6,6′-tetrasulfonic acid, phenylene-1,4-bis(monosodium 2-benzimidazolyl-5,7-disulfonic acid) or 3,3′-(1,4-phenylenedimethylidene)bis(7,7-dimethyl-2-oxobicyclo[2,2,I ]heptane-1-methanesulfonic acid) and salts thereof], salicylates (e.g. 2-ethylhexyl salicylate or homomenthyl salicylate), triazines {e.g. 2,4-bis[4-(2-ethylhexyloxy)-2-hydroxyphenyl]-6-(4-methoxyphenyl)-1,3,5-triazine, bis(2-ethylhexyl)4,4′-([6-([(1,1-dimethylethyl)aminocarbonyl]phenylamino)-1,3,5-triazine-2,4-diyl]diimino)bisbenzoate, 2-cyanopropenoic acid derivatives (e.g. 2-ethylhexyl 2-cyano-3,3-diphenyl-2-propenoate), dibenzoyl derivatives (e.g. 4-tert-butyl-4′-methoxydibenzoylmethane), polymer-bonded UV filters (e.g. polymers of N-[2-(or 4)-(2-oxo-3-bornylidene)methyl]benzylacrylamide) or pigments (e.g. titanium dioxides, zirconium dioxides, iron oxides, silicon dioxides, manganese oxides, aluminum oxides, cerium oxides or zinc oxides).

The lipid phase in the cosmetic or dermatological preparations can advantageously be chosen from the following group of substances: mineral oils (advantageously paraffin oil), mineral waxes, hydrocarbons (advantageously squalane or squalene), synthetic or semisynthetic triglyceride oils (e.g. triglycerides of capric acid or caprylic acid), natural oils (e.g. castor oil, olive oil, sunflower oil, soybean oil, peanut oil, rapeseed oil, almond oil, palm oil, coconut oil, palm kernel oil, borage seed oil and the like), natural ester oils (e.g. jojoba oil), synthetic ester oils (preferably esters of saturated and/or unsaturated, linear and/or branched alkanecarboxylic acids having 3 to 30 carbon atoms with saturated and/or unsaturated, linear and/or branched alcohols having 3 to 30 carbon atoms and esters of aromatic carboxylic acids with saturated and/or unsaturated, linear and/or branched alcohols having 3 to 30 carbon atoms, in particular chosen from the group isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laureate, 2-hexyldecyl stearate, 2-octyldecyl palmitate, oleyl oleate, oleyl erucate, erucyl oleate, erucyl erucate, and synthetic or natural mixtures of such esters), fats, waxes and other natural and synthetic fatty substances, preferably esters of fatty alcohols with alcohols of low carbon number (e.g. with isopropanol, propylene glycol or glycerol) or esters of fatty alcohols with alkanoic acids of low carbon number or with fatty acids, alkyl benzoates (e.g. mixtures of n-dodecyl, n-tridecyl, n-tetradecyl and n-pentadecyl benzoate), and cyclic or linear silicone oils (such as, for example, dimethylpolysiloxanes, diethylpolysiloxanes, diphenylpolysiloxanes, and mixed forms thereof).

The aqueous phase in the cosmetic or dermatological preparations optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl ether, monoethyl ether or monobutyl ether, diethylene glycol monomethyl or mono-ethyl ether and analogous products, and also alcohols of low carbon number, e.g. ethanol, isopropanol, 1,2-propanediol, glycerol, and also α- or β-hydroxy acids, preferably lactic acid, citric acid or salicylic acid, and also emulsifiers which may advantageously be chosen from the group of ionic, nonionic, polymeric, phosphate-containing and zwitterionic emulsifiers, and in particular one or more thickeners which may advantageously be chosen from the group silicon dioxide, aluminum silicates, such as, for example, bentonites, polysaccharides or derivatives thereof, e.g. hyaluronic acid, guar seed flour, xanthan gum, hydroxypropylmethylcellulose or allulose derivatives, particularly advantageously from the group of polyacrylates, preferably a polyacrylate from the group of Carbopols, in each case individually or in combination, or from the group of polyurethanes.

The present invention likewise also encompasses a method of protecting cosmetic or pharmaceutical, preferably dermatological, preparations, and foods against oxidation or photooxidation, the constituents of which are associated with stability problems based on oxidation or photooxidation during storage, characterized in that the preparations have an effective content of 3,4-dihydroxymandelic acids. The use according to the invention can of course also be used analogously in other fields of use, e.g. for protecting surface coatings or technical-grade polymers.

The amount of 3,4-dihydroxymandelic acids in the cosmetic or pharmaceutical, preferably dermatological, preparations and in foods is 0.001% by weight to 1% by weight, preferably 0.001% by weight to 0.5% by weight, based on the total weight of the preparations.

### EXAMPLES

### Example 1

### Activity as Free-radical Scavenger

The activity of 3,4-dihydroxymandelic acid as free-radical scavenger was compared with that of conventional free-radical scavengers. For this purpose, the 1,1-diphenyl-2-picrylhydrazyl test (DPPH test) for the removal of free radicals was used.

1,1-Diphenyl-2-picrylhydrazyl (DPPH) was dissolved in methanol to a concentration of 100 μmol/l. A series of dilutions of 3,4-dihydroxymandelic acid, vitamin C, α-tocopherol and 2,6-di-tert-butyl-4-methylphenol were prepared in methanol. Methanol was used as the control. 2500 μl of the DPPH solution were mixed with 500 μl of each test solution and the decrease in absorption at 515 nm was read until the decrease was less than 2% per hour. The activity of the test substances as free-radical scavengers was calculated using the following equation:

Activity as free-radical scavenger (%)=100−(absorption of the test compounds)/(absorption of the control)×100.

The activity of the free-radical scavengers (%) in a series of dilutions of test compounds was used to calculate, for each test compound, the effective relative concentration EC₅₀ (based on the starting concentration of DPPH, EC=c (test compound)/c(DPPH)) of a test compound at which 50% of the free radical DPPH had been removed. The results are given in Table 1:

TABLE 1

| Test compound | CAS No. | EC₅₀/(mol/mol) |
|---|---|---|
| 3,4-Dihydroxymandelic acid | 14883-87-5 | 0.062 |
| Vitamin C | 50-81-7 | 0.270 |
| α-Tocopherol | 59-02-9 | 0.250 |
| 2,6-Di-tert-butyl-4-methylphenol | 128-37-0 | 0.240 |

### Example 2

### Activity as Antioxidant

The activity of 3,4-dihydroxymandelic acid as antioxidant was compared with that of conventional antioxidants. The test system used was the accelerated autoxidation of lipids by air with or without antioxidant using the Rancimat apparatus (Rancimat is a registered trademark of Metrohm AG, Herisau, Switzerland).

3,4-Dihydroxymandelic acid, vitamin C, α-tocopherol and 2,6-di-tent-butyl-4-methylphenol were dissolved in methanol or acetone, and 100 μl of each test solution were added to a 3 g prepared oil sample. In a control sample, only solvent was added. A constant dry stream of air (20 l/h) was bubbled through the heated oil sample, which contained the test solution, and the volatile oxidation products (predominantly short-chain fatty acids such as formic acid or acetic acid) were collected in a receiver containing water. The conductivity of this aqueous solution was continuously measured and documented. The oxidation of (unsaturated) fats proceeds only very slowly for some time and then suddenly increases. The time to the increase is referred to as the induction period (IP).

The following equation was used to calculate the antioxidative index (AOI):

AOI=IP_{(with test solution}/IP_{(control sample)}

The results for the experiment in soybean oil that has been purified over alumina grade N at 100° C. are shown in Table 2:

TABLE 2

| | AOI in soybean oil |
|---|---|
| | at 100° C. with |
| Test compounds | 0.05% test substance |
| 3,4-Dihydroxymandelic acid | 9.5 |
| Vitamin C | 1.2 |
| α-Tocopherol | 5.1 |
| 2,6-Di-tert-butyl-4-methylphenol | 4.8 |

The results for the experiment in squalene that has been purified over alumina grade N and stabilized with 1 ppm of α-tocopherol at 80° C. are shown in Table 3:

TABLE 3

| | AOI in squalene |
|---|---|
| | at 80° C. with 0.005% |
| Test compounds | of test substance |
| 3,4-Dihydroxymandelic acid | 34 |
| Vitamin C | 0.7 |
| α-Tocopherol | 39 |
| 2,6-Di-tert-butyl-4-methylphenol | 38 |

### Example 3

### Antioxidative Action in Combination with Tocopherol

The experiments were carried out as described under Example 2. In this case, the AOI was measured in each case in soybean oil at 100° C. for 0.025% of 3,4-dihydroxymandelic acid, 0.025% of tocopherol and for a combination of 0.025% of 3,4-dihydroxymandelic acid and 0.025% of tocopherol, and the AOI was measured in each case in squalene at 80° C. for 0.0025% of 3,4-dihydroxymandelic acid, 0.0025% of tocopherol and for a combination of 0.0025% of 3,4-dihydroxymandelic acid and 0.0025% of tocopherol. The AOI values measured for the combinations were compared with the AOI values calculated according to the following formula:

AOI_{combination, calculated}=(AOI_{3,4}-dihydroxymandelic acid−1)+(AOI_{tocopherol}−1)+1

TABLE 4

| | | AOI, | | |
|---|---|---|---|---|
| | | soybean | | AOI, |
| | c (% | oil, | c (% | squalene, |
| Test compound | by wt.) | 100° C. | by wt.) | 80° C. |
| 3,4-Dihydroxymandelic acid | 0.025 | 5.4 | 0.0025 | 15 |
| α-Tocopherol | 0.025 | 4.7 | 0.0025 | 23 |
| 3,4-Dihydroxymandelic acid + | 0.05 | 17 | 0.005 | 54 |
| α-tocopherol (1:1) | | | | |
| 3,4-Dihydroxymandelic acid + | 0.05 | calculated: | 0.05 | calculated: |
| α-tocopherol (1:1) | | 9.1 | | 37 |

### Example 4

### Determination of the Protective Action against Ultraviolet Light-induced Sebum Oxidation

200 mg of 3,4-dihydroxymandelic acid were dissolved in 200 ml of 1,3-butylene glycol (solution A). In addition, a 0.2% strength ethanolic solution of tocopherol was prepared (solution B). A dose of 2 mg/cm² of solution A was applied twice daily to the skin on the back of 12 subjects for 2 days in each case. Prior to the following irradiation, solution B was applied to a control area (2 mg/cm²). The 2 treated and one untreated sites were irradiated with ultraviolet light (320 to 400 nm, 10 joules/cm²). The test areas in each case were treated with 4 ml of ethanol for 2 min, the solutions were dried under nitrogen at room temperature and the residue was taken up in 1 ml of ethanol. The latter solutions were investigated by HPLC for their content of squalene (detection at 210 nm against standard) or squalene hydroperoxide (SQOOH, determination of the peroxide content using cytochrome C/luminol-enhanced chemiluminescence). The content of squalene peroxide was given relative to squalene in the form of picomols peroxide per fig of squalene.

The inhibition based on the untreated area was calculated using the following equation:

% inhibition=100·(c_{SQOOH untreated}−c_{SQOOH, treated})/C_{SQOOH, untreated}

TABLE 5

| | c(H₂O₂)/c(SQ) | % inhibition compared |
|---|---|---|
| Test compound | [pmol/μg] | with the untreated area |
| Untreated area | 930 ± 65 | — |
| 3,4-Dihydroxymandelic acid | 618 ± 45 | 33 ± 7 |
| Tocopherol | 664 ± 19 | 28 ± 6 |

The examples below serve to illustrate the present invention without limiting it:

### Example 6

### “Oil-in-water” Emulsion Comprising 3,4-dihydroxymandelic Acid

| | Raw material name | | Content in |
|---|---|---|---|
| Part | (manufacturer) | Chemical name | % by wt. |
| A | Arlatone 983 S ® (ICI) | Ether of polyethylene | 1.2 |
| | | glycol with glycerol | |
| | | monostearate | |
| | Brij 76 ® (ICI) | 3,6,9,12,15,18,21,24,27,30, | 1.2 |
| | | 33,36-Decaoxaoctatetracon- | |
| | | tan-1-ol | |
| | Cutina MD ® (Henkel) | Glyceryl monostearate | 3.5 |
| | Baysiloneöl M10 ® | Polydimethylsiloxane | 0.8 |
| | (GE Bayer) | | |
| | Eutanol G ® (Henkel) | Octyldodecanol | 3.0 |
| | Paraffin oil 65 cp | Mineral oil | 8.0 |
| | (Henry Lamotte) | | |
| B | Water, dist. | | 50.35 |
| | Phenopip ® | 2-Phenoxyethanol and | 0.5 |
| | (Nipa Laboratories) | methyl 4-hydroxybenzoate | |
| | | and ethyl | |
| | | 4-hydroxybenzoate and | |
| | | propyl 4-hydroxybenzoate | |
| | | and butyl | |
| | | 4-hydroxybenzoate | |
| | 1,2-Propylene glycol | | 2.0 |
| | Glycerol 99% | | 3.0 |
| | Trilon ® BD (BASF) | Disodium ethylenediamine- | 0.1 |
| | | tetraacetic acid | |
| | 3,4-Dihydroxymandelic | | 0.1 |
| | acid | | |
| C | Water, dist. | | 25.0 |
| | Carbopol 2050 ® | Crosslinked acrylic acid/ | 0.4 |
| | (B. F. Goodrich) | C₁₀-C₃₀-alkyl acrylate | |
| | | polymer | |
| | Aqueous sodium | | 0.85 |
| | hydroxide soln., 10% | | |

Part A was mixed and heated to 80° C. Part B was mixed and heated to 90° C. and added to Part A with stirring. For Part C, Carbopol was carefully dispersed in water and neutralized with sodium hydroxide solution (pH 5.3). Part C was then added to the mixture of Parts A and B at 60° C.

### Example 7

### “Oil-in-water” Emulsion Comprising 3,4-dihydroxymandelic Acid and α-tocopherol

| | Raw material name | | Content in |
|---|---|---|---|
| Part | (manufacturer) | Chemical name | % by wt. |
| A | Arlatone 983 S ® (ICI) | Ether of polyethylene | 1.2 |
| | | glycol with glycerol | |
| | | monostearate | |
| | Brij 76 ® (ICI) | 3,6,9,12,15,18,21,24,27,30, | 1.2 |
| | | 33,36-Decaoxaoctatetracon- | |
| | | tan-1-ol | |
| | Cutina MD ® (Henkel) | Glyceryl monostearate | 3.5 |
| | Baysiloneöl M10 ® | Polydimethylsiloxane | 0.8 |
| | (GE Bayer) | | |
| | Eutanol G ® (Henkel) | Octyldodecanol | 3.0 |
| | Paraffin oil 65 cp | Mineral oil | 7.9 |
| | (Henry Lamotte) | | |
| | α-Tocopherol | | 0.1 |
| B | Water, dist. | | 50.45 |
| | Phenopip ® | 2-Phenoxyethanol and | 0.5 |
| | (Nipa Laboratories) | methyl 4-hydroxybenzoate | |
| | | and ethyl | |
| | | 4-hydroxybenzoate and | |
| | | propyl 4-hydroxybenzoated | |
| | | and butyl | |
| | | 4-hydroxybenzoate | |
| | 1,2-Propylene glycol | | 2.0 |
| | Glycerol 99% | | 3.0 |
| | 3,4-Dihydroxymandelic | | 0.1 |
| | acid | | |
| C | Water, dist. | | 25.0 |
| | Carbopol 2050 ® | Crosslinked acrylic acid/ | 0.4 |
| | (B. F. Goodrich) | C₁₀-C₃₀-alkyl acrylate | |
| | | polymer | |
| | Aqueous sodium | | 0.85 |
| | hydroxide soln., 10% | | |

Part A was mixed and heated to 80° C. Part B was mixed and heated to 90° C. and added to Part A with stirring. For Part C, Carbopol was carefully dispersed in water and neutralized with sodium hydroxide solution (pH 5.3). Part C was then added to the mixture of Parts A and B at 60° C.

### Example 8

### “Oil-in-water” Emulsion with UVA/B Broadband Protection, 3,4-dihydroxymandelic Acid and α-tocopherol

| | Raw material name | | Content in |
|---|---|---|---|
| Part | (manufacturer) | Chemical name | % by wt. |
| A | Crodaphos MCA ® (Croda) | Cetyl phosphate | 1.50 |
| | Cutina MD ® (Henkel) | Glyceryl stearate | 2.0 |
| | Lanette 16 ® (Henkel) | Cetyl alcohol | 1.2 |
| | Myritol 318 ® (Henkel) | Caprylic/caproic | 5.0 |
| | | triglycerides | |
| | Cetiol SN ® (Henkel) | Cetyl and stearyl | 5.0 |
| | | isononanoate | |
| | α-Tocopherol | | 0.1 |
| | Solbrol P ® (Bayer) | Propyl | 0.1 |
| | | 4-hydroxybenzoate | |
| | Abil 100 ® (Goldschmidt) | Polydimethylsiloxane | 0.3 |
| | Neo Heliopan ® HMS | 3,3,5-Trimethylcyclo- | 5.0 |
| | (Haarmann & Reimer) | hexyl salicylate | |
| | Neo Heliopan ® 357 | Butylmethoxydi- | 2.0 |
| | (Haarmann & Reimer) | benzoylmethane | |
| B | Water, dist. | | 48.3 |
| | 1,3-Butylene glycol | | 3.0 |
| | Sobrol M ® (Bayer) | Methyl | 0.2 |
| | | 4-hydroxybenzoate | |
| | Phenoxyethanol | | 0.7 |
| | Carbopol ETD 2050 ® | Copolymer of acrylic | 0.2 |
| | (B. F. Goodrich) | acid/C₁₀-C₃₀-alkyl | |
| | | acrylate | |
| | Keltrol T ® (Calgon) | Xanthan gum | 0.2 |
| | Neo Heliopan ® AP | 2,2-(1,4-Phenylene)bis | 22 |
| | (Haarmann & Reimer) | (1H-benzimidazole- | |
| | | 4,6-disulfonic acid) | |
| | | and disodium salt | |
| | 3,4-Dihydroxymandelic acid | | 0.1 |
| C | Aq. sodium hydroxide | | 2.8 |
| | soln., 10% | | |
| D | Perfume oil | | 0.3 |

Part A was heated to 85° C. Part B: Carbopol and Keltrol were dispersed into the remaining constituents while cold, the mixture was heated to 85° C. and added to Pail A. Part C was immediately added, at 80° C., to the mixture of Parts A and B and homogenized for 5 min using a dispersing tool. Part D was finally added at room temperature and the mixture was homogenized using a dispersing tool (pH 6.6).

1. The use of 2-(3,4-dihydroxyphenyl)-2-hydroxyacetic acid including its stereoisomers or mixtures thereof for protecting the skin of mammals against harmful oxidative processes.

2. The use of cosmetic or dermatological preparations comprising 0.001% by weight to 1% by weight, based on the total weight of the preparations, of 2-(3,4-dihydroxyphenyl)-2-hydroxyacetic acid, stereoisomers thereof or mixtures thereof for protecting the skin of mammals against harmful oxidative processes.

3. The use of 2-(3,4-dihydroxyphenyl)-2-hydroxyacetic acid including its stereoisomers or mixtures thereof in combination with further antioxidants or free-radical scavengers for protecting the skin of mammals against harmful oxidative processes.

4. The use of cosmetic or dermatological preparations as claimed in claim 2, additionally comprising 0.001% by weight to 10% by weight, based on the total weight of the preparations, of further antioxidants and/or free-radical scavengers.

5. The use as claimed in claims 3 and 4, characterized in that further antioxidants and/or free-radical scavengers are antioxidants suitable for cosmetic and/or dermatological applications.

6. The use as claimed in claims 1 to 5, characterized in that the harmful oxidative processes are induced by ultraviolet light.

7. The use as claimed in claims 1 to 6, characterized in that the protection of the skin of mammals includes the protection of the extra- and intracellular skin lipids.

8. The use of 2-(3,4-dihydroxyphenyl)-2-hydroxyacetic acid including its stereoisomers or mixtures thereof for protecting cosmetic preparations against oxidation or photooxidation.

9. The use of 2-(3,4-dihydroxyphenyl)-2-hydroxyacetic acid including its stereoisomers or mixtures thereof for protecting pharmaceutical preparations against oxidation or photooxidation.

10. The use of 2-(3,4-dihydroxyphenyl)-2-hydroxyacetic acid including its stereoisomers or mixtures thereof for protecting foods against oxidation or photooxidation.

11. The use of 2-(3,4-dihydroxyphenyl)-2-hydroxyacetic acid including its stereoisomers or mixtures thereof for protecting surface coatings against oxidation or photooxidation.

12. The use of 2-(3,4-dihydroxyphenyl)-2-hydroxyacetic acid including its stereoisomers or mixtures thereof for protecting polymers against oxidation or photooxidation.

13. The use of 2-(3,4-dihydroxyphenyl)-2-hydroxyacetic acid including its stereoisomers or mixtures thereof as claimed in claims 8 to 12 in combination with at least one further antioxidant or free-radical scavenger.
